(19) United States
(12) Patent Application Publication
(10) Pub. No.: US 2025/0332386 A1
(43) Pub. Date: Oct. 30, 2025

(54) WIRE MANAGEMENT DEVICE WITH GUIDEWIRE COMPARTMENTALIZATION

(71) Applicant: Rumi Faizer, Claremont, CA (US)

(72) Inventor: Rumi Faizer, Claremont, CA (US)

(21) Appl. No.: 18/649,691

(22) Filed: Apr. 29, 2024

(51) Int. Cl. A61M 25/06

(52) U.S. Cl. CPC A61M 25/0668

(57) ABSTRACT

A wire management device including a sheath, a plurality of divider assemblies or partitions, and a valve assembly. The divider assemblies project inwardly from an inner surface of the sheath, and collectively divide a lumen of the sheath into two or more channels that are each sized to slidably receive a guidewire. The divider assemblies are collapsible from a normal, extended state to a collapsed state. The valve assembly serves to selectively close or fluidly seal respective ones of the channels at a proximal end of the sheath. Individual guidewires can be advanced through corresponding ones of the channels via the valve assembly. A delivery device can be loaded to the guidewires and advanced through the sheath causing the divider assemblies are caused to collapse. Ahead of the delivery device, the divider assemblies remain extended, and thus the guidewires remain separated and unable to tangle.

### CROSS-REFERENCE TO RELATED APPLICATIONS

This Non-Provisional patent application claims the benefit of the filing date of U.S. Provisional Patent Application No. 63/462,687, filed Apr. 28, 2023, the entire teachings of which are incorporated herein by reference

### BACKGROUND

The present disclosure relates to catheter and guidewire systems. More particularly, it relates to catheter-based systems employing two or more guidewires to deliver to a therapeutic device to a patient.

A variety of different therapies can be delivered within the human body by catheter systems or devices. Therapeutic devices such as stents, stent grafts, endografts, filters, dilation balloons are but a few examples, and are conventionally delivered to a target site in a contracted or compressed state within a catheter. The device is typically loaded at a distal end of the catheter; once properly located, the catheter is proximally retracted and/or the device is distally advanced.

To aid in positioning of the distal end of the catheter within the body, typically a guidewire is first navigated to the treatment area. After the guidewire has been positioned, the catheter can be directed along or over the guidewire, bringing the distal end of the catheter to a desired position. In this regard, the catheter will form at least one lumen that slidably receives the guidewire. For many procedures, the catheter will provide two (or more lumens), with at least one of the lumens dedicated to the guidewire.

In addition to facilitating catheter placement, guidewires are also employed to achieve desired arrangement or deployment of the catheter-delivered device in some instances. For example, devices intended to branch across bodily vessel bifurcation (e.g., a bifurcated stent graft). In vessel bifurcations, a main vessel splits into two branch vessels. Implanting stents or stent grafts in bifurcations is particularly problematic because of the need to precisely locate the stent both longitudinally and radially in the bifurcation, for example to locate a side opening or branch of the stent graft to face and extend into the branching vessel. Such devices and corresponding methods of delivery require complicated manipulations and precise delivery to specific target locations. Where the stent graft or other device to be implanted provides multiple side openings (fenestrations) or branches each intended to face or be located within a separate branch vessel (e.g., an abdominal aortic aneurysm stent graft), the procedure is even more complicated. Oftentimes, multiple guidewires are required to properly align each opening with respect to a corresponding branch vessel.

Where a particular procedure benefits from the provision of multiple guidewires to effectuate alignment of the device to be implanted relative to the native anatomy, clinicians prefer that the multiple guidewires be delivered through a single catheter (as opposed to providing a separate catheter and access approach for each guidewire). While loading of the guidewires to the catheter or delivery sheath and subsequent delivery of the device over the guidewires once in place is in theory straightforward, problems may arise. In particular, due the tortuous delivery path presented by many procedures and/or the manipulations of the guidewires in order to achieve necessary vessel or side branch location, two or more of the guidewires oftentimes wrap or twist about one another. Wrapping of the guidewires can be highly problematic as the device cannot then be readily advanced over the guidewires.

Existing strategies to deal with the need to stent across branch points in the aorta largely resort to modular techniques. This is complex to deploy and leads to long-term durability risks. The challenge has been an issue termed wire wrap: once two or more wires coexist in the aorta, one will tend to wrap around the other. As the device is advanced over the wires it will twist to follow the helical path induced by the wrap of the wires, ending with an incorrectly oriented device.

### SUMMARY

The inventor of the present disclosure recognizes that a need exists for a catheter system that overcomes one or more of the above-mentioned problems.

Some aspects of the present disclosure provide a wire management device that presents a definitive solution to wire wrap which has been demonstrated to allow a branched graft to be relatively easily advanced to its final destination over multiple pre-placed wires.

Some embodiments of the present disclosure include a wire management device including a sheath with a lumen that is separated by longitudinal partitions or divider assemblies into two or more (e.g., four) separate channels and that can be collapsed into a single channel was created. The partitions run the length of the sheath and are attached to the sheath wall, protruding inward and meeting at the center of the sheath. Viewed from end on they therefore appear to be spokes of a wheel. The partitions are designed like the vanes of a feather, allowing them to collapse completely against the sheath wall when compressed from the proximal side (the side of the sheath valve). Like the vane of a feather, however, the portions that are not being pushed will stay extended. In this regard, pushing the vanes from below is akin to “unzipping” the partitions. As with a zipper that can only be opened from the end, once inside a channel the vanes cannot be collapsed or separated and so keep the compartments separate. The valve for the sheath also has four leaflets but their partitions are turned 45 degrees from the vanes such that when a wire is passed between the valve leaflets it enters into the center (lumen) of a partition of the sheath.

During use, guidewires are passed separately down each of the channels and then directed by the operator under fluoroscopy into the different visceral branches. The back end of each wire is used to cannulate the corresponding opening in a constrained branched or fenestrated aortic endograft, for example. The constrained endograft is then advanced successfully through the sheath, first collapsing the valve system forward (into the sheath), and then collapsing the vanes of the partitions in the sheath thereby unifying the lumen. The constrained endograft can thus be advanced up the sheath collapsing the vanes and uniting the lumens as it advances (“unzipping” the partitions) until it reaches the end of the sheath. Because the vanes stay erect until the endograft collapses them from below, the wires stay separated until the point where the endograft reaches them. With the sheath positioned right at the lowest visceral branch, once the device is at the end of the sheath, the sheath can be pulled back and the device unconstrained partially to allow separation of each of its branches (or exposure of the fenestrations). The device can then be cautiously advanced forward until each of its branches has advanced to the origin of the corresponding visceral branch-its final destination. The endograft can then be completely opened. Bridging stents as necessary can be advanced over each visceral branch wire to make a complete seal. With the endograft deployed the delivery system is retrieved. After ballooning of the stents to ensure seal, the wires can be removed. Lastly, the wire management device is removed. As a result, the endograft is completely deployed and wire-wrap is completely averted with the wire management devices of the present disclosure.

### DETAILED DESCRIPTION

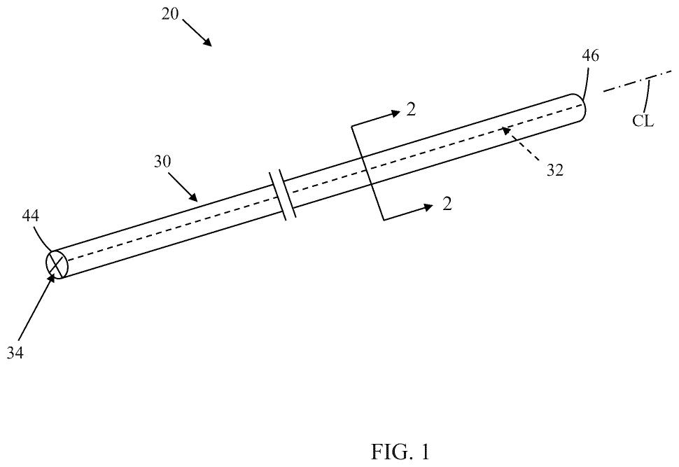

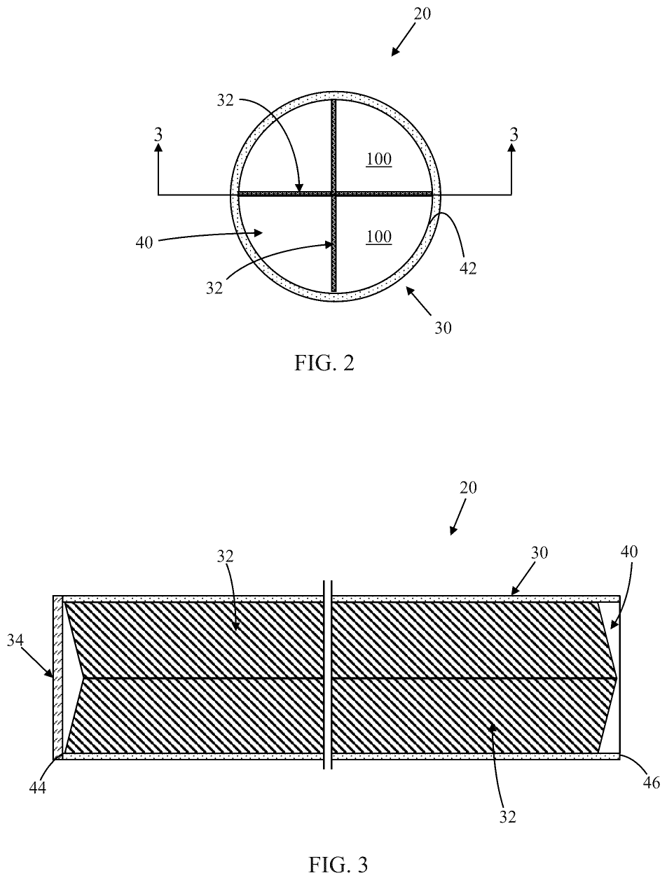

One embodiment of a wire management device 20 in accordance with principles of the present disclosure is shown in FIGS. 1-3. The wire management device 20 includes a sheath or catheter 30, a plurality of divider assemblies or partitions 32, and a valve assembly 34. Details on the various components are provided below. In general terms, divider assemblies 32 project inwardly from an inner surface of the sheath 30, and collectively divide a lumen of the sheath 30 into two or more channels that are each sized to slidably receive a guidewire (not shown). The divider assemblies 32 are collapsible from a normal, extended state (reflected by FIGS. 2 and 3) to a collapsed state within the sheath 30. The valve assembly 34 serves to selectively close or fluidly seal respective ones of the channels at a proximal end of the sheath 30. With this construction, individual guidewires can be advanced through corresponding ones of the channels via a segment of the valve assembly 34, with the divider assemblies 32 serving to compartmentalize or separate the so-arranged guidewires. Once the guidewires have been arranged as desired relative to native anatomy, a delivery device (e.g., endograft delivery device) can be loaded to the guidewires and advanced through the sheath 30. As the delivery device is advanced, the divider assemblies 32 are caused to collapse, allowing the delivery device to be directed as desired. However, ahead of or distal the delivery device, the divider assemblies 32 remain in the extended state, and thus the guidewires remain separated from one another and unable to tangle.

The sheath 30 can assume a wide variety of forms appropriate for accessing and traversing a bodily lumen (or lumens) of a human patient. Thus, the delivery sheath 30 can be akin to a conventional catheter (e.g., a biologically compatible tube with sufficient column strength for traversing tortuous anatomy that may optionally incorporate steering features), having a tubular construction defining a lumen 40 that is circumscribed by an inner surface 42 of the sheath. The lumen 40 extends between opposing, proximal and distal ends 44, 46 of the sheath 30.

The divider assemblies 32 can assume a wide variety of forms conducive to providing the extended and contracted states as described below. While FIG. 2 illustrates the wire management device 20 as including four of the divider assemblies 32, any other number, either greater or lesser, is equally acceptable (e.g., in other embodiments, six of the divider assemblies 32 are provided). The wire management devices of the present disclosure include at least two of the divider assemblies 32.

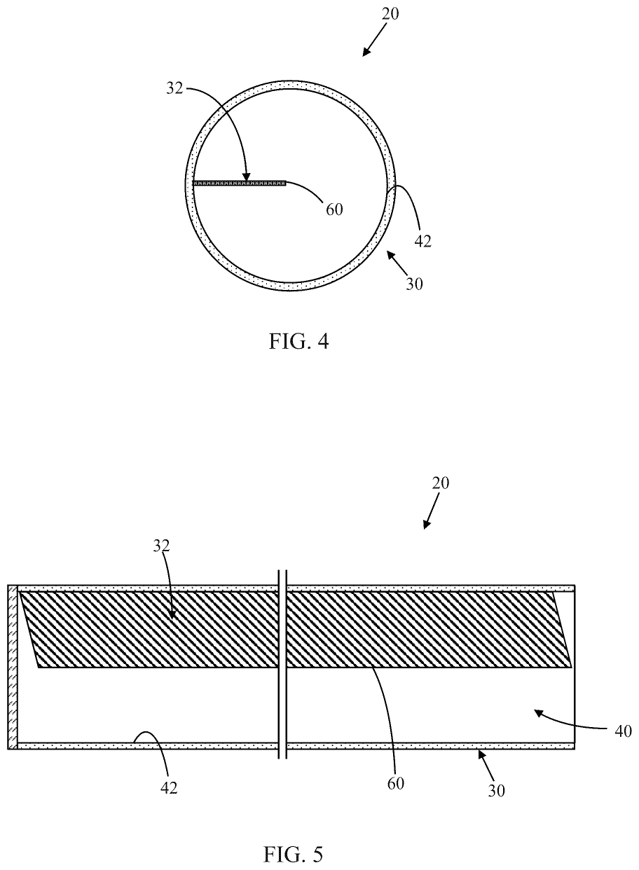

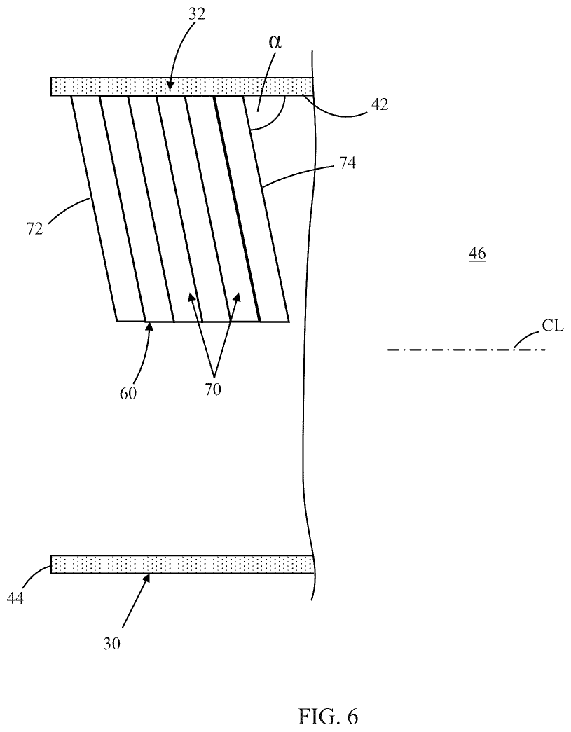

The divider assemblies 32 each project in a radial fashion (relative to a centerline CL of the sheath 30) from the inner surface 42 of the sheath 30 (and thus within the lumen 40), and are circumferentially spaced from one another about a circumference of the inner surface 42. With additional reference to FIGS. 4 and 5 that are otherwise identical to the views of FIGS. 2 and 3, respectively, except that all but one of the divider assemblies 32 are removed for ease of explanation, each of the divider assemblies 32 terminates at contact edge 60 opposite the inner surface 42. The divider assemblies 32 are sized and shaped such that in the normal, extended state, the contact edges 60 interface with or overlap one another within the lumen 40 at an approximate center thereof as shown in FIGS. 2 and 3. In this arrangement and when viewed from an end of the sheath 30 (e.g., the view of FIG. 2), the divider assemblies 32 appear to be spokes of a wheel. The divider assemblies 32 can have an identical construction in some embodiments. FIG. 6 illustrates a portion of a one of the divider assemblies 32 in enlarged form; the divider assembly 32 includes or consists of a plurality of vanes or bristles 70. The vanes 70 can be longitudinally aligned with one another (e.g., aligned parallel to the centerline CL of the sheath 30), and collectively define the corresponding contact edge 60. In some embodiments, the vanes 70 are closely positioned relative to each other, collectively forming a membrane-like or barrier-like structure (e.g., the vanes 70 are confluent and effectively create a membrane due to close apposition of the vanes 70 so as to, for example, prevent a guidewire from passing “through” the divider assembly 32). In other embodiments, the divider assemblies 32 can each have a homogenous or integral structure. In yet other embodiments, a slight spacing can exist between immediately adjacent ones of the vanes 70.

The divider assemblies 32 can, in some embodiments, be formed of a biocompatible material (e.g., PTFE or similar material). A material and construction of the divider assemblies 32 is selected to have sufficient rigidity to prevent a guidewire from passing (in a radial direction) through the line of intersection or apposition between the divider assemblies 32. However, the divider assemblies 32 are each configured to readily deflect or collapse from the extended state to the collapsed state in response to a longitudinally-applied force, for example an endograft delivery system being distally advanced over guidewires (not shown) carried by the wire management device 20 as described in greater detail below.

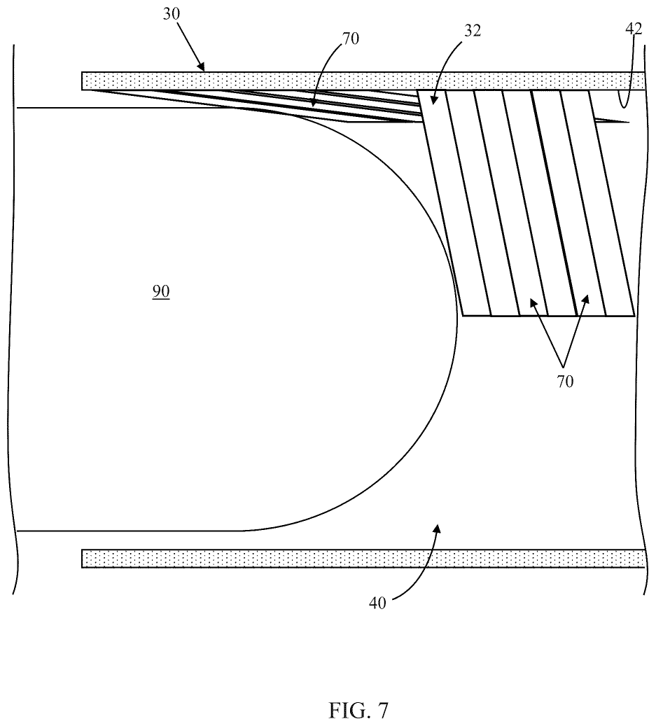

In some optional embodiments, the divider assemblies 32 are each configured to incorporate a shape bias in the distal direction. For example, the plurality of vanes 70 collectively defines the divider assembly 32 to have a proximal side 72 opposite a distal side 74. Each side 72, 74 projects from the inner surface 42 of the sheath 30 to the contact edge 60, with this projection defining a bias angle α (best seen and identified in FIG. 6). In the extended state of FIG. 6, the bias angle α can be an acute angle, for example in the range of 25°-85°, and faces or is open to the distal end 46 (identified generally in FIG. 6) of the sheath 30. A similar relationship can be established at the proximal side 72. Each of the vanes 70 can exhibit this same geometry. For example, each of the vanes 70 can have a shape akin to a parallelogram, and are arranged on the inner surface 42 so as to be skewed in the distal direction. With this construction, the consecutive ones of the vanes 70 will readily collapse toward the inner surface 42 when contacted by a body being distally advanced through the lumen 40. For example, FIG. 7 illustrates a delivery device 90 (e.g., an endograft delivery device) inserted into the lumen 40 and being advanced in the distal direction; as individual vanes 70 of the divider assembly 32 are contacted by the delivery device 90, they readily collapse toward the inner surface 42, for example due to the shape bias described above. Regardless, as the vanes 70 collapse, the delivery device 90 can continue to easily move in the distal direction. However, the vanes 70 distal the delivery device 90 (and thus not yet contacted by the delivery device 90) remain in the extended state. The divider assemblies 32 can have other constructions that provide these same features that may or may not include a plurality of vanes or bristles or similarly-shaped bodies.

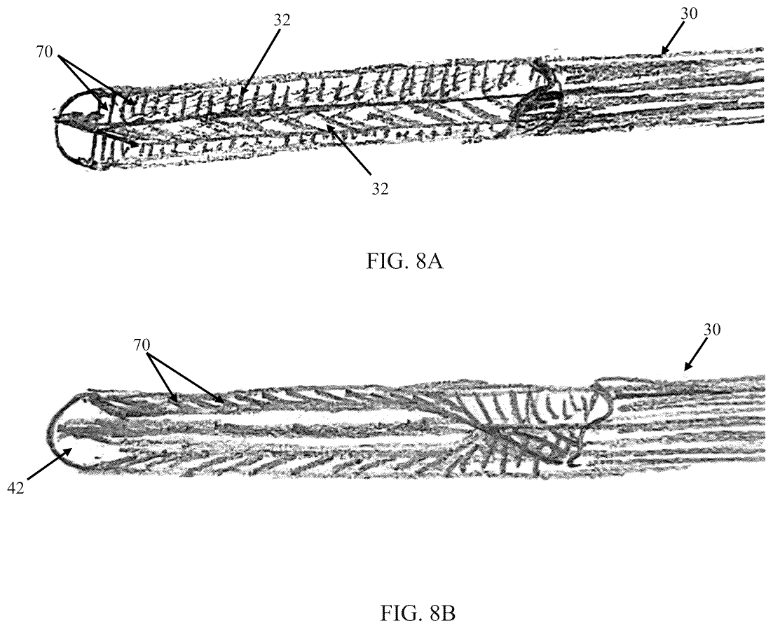

This feature is further reflected by a comparison of the simplified cut-away views of FIGS. 8A and 8B. FIG. 8A illustrates the divider assemblies 32 in the normal, extended state. FIG. 8B reflects collapsing of the divider assemblies 32 (e.g., the individual vanes 70 of each of the divider assemblies 32 have been forced to collapse toward the inner surface 42 of the sheath 30.

Returning to FIG. 6, with embodiments in which the divider assemblies 32 are formed by the plurality of vanes 70 as described above, the divider assemblies 32 are feather-like or akin to a feather. If one runs one's finger up the shaft of a feather, the vanes of the feather will collapse and flatten with little force. The vanes of the feather, however, provide sufficient surface tension to function as a fan; the barbs carried by each vane tend to stick together to create a membrane that prevents even air from escaping across it.

With reference to FIG. 2, in the normal, extended state, the divider assemblies 32 contact or interface with one another at the corresponding contact edges 60, compartmentalize the lumen 40 into two or more channels 100 (e.g., with the embodiment of FIG. 2, four of the channels 100 are provided). The channels 100 are separated from one another by the corresponding divider assemblies 32, and are open to the distal end 46 (FIG. 3) of the sheath 30. The channels 100 run the length of the sheath 30 to the proximal end 44 at which the valve assembly 34 provides for selective access to the channels 100 as described below.

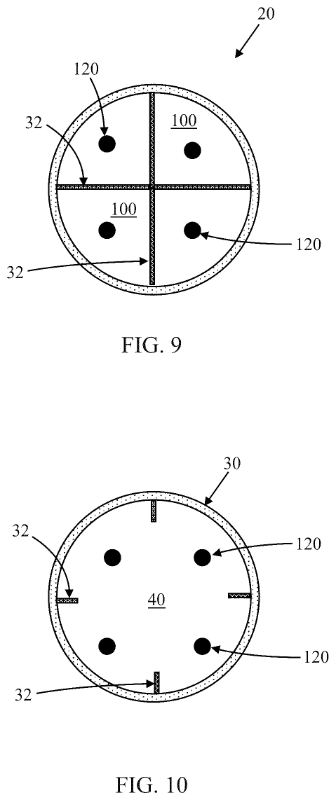

The channels 100 are sized and shaped to slidably receive at least one guidewire. For example, FIG. 9 shows use of the wire management device 20 with a plurality of guidewires 120, with each guidewire 120 slidably disposed within a respective one of the channels 100. In the extended state, the divider assemblies 32 isolate the guidewires 120 from one another, with each guidewire remaining within the corresponding channel 100. In this compartmentalized arrangement, the guidewires 120 are prevented from becoming tangled or experiencing wire-wrap. However, when the divider assemblies 32 are forced to the collapsed state, as generally reflected by FIG. 10, the discrete channels 100 are effectively obliterated, effectively unifying the lumen 40.

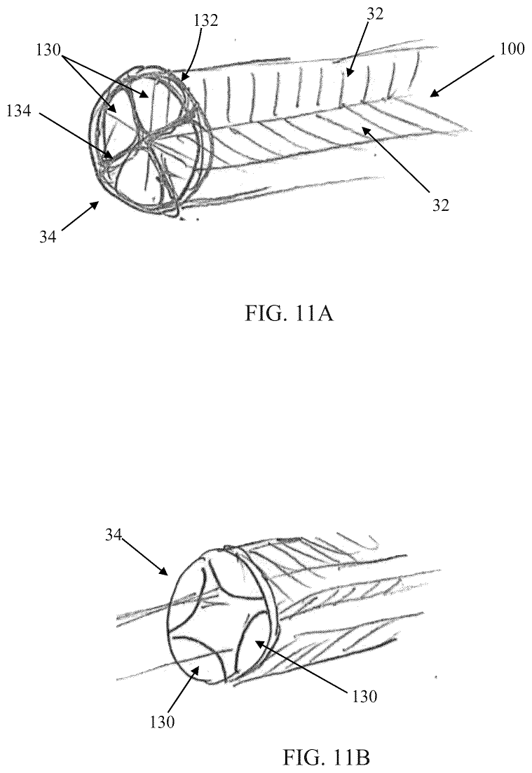

Returning to FIGS. 1-3, the valve assembly 34 fluidly closes the channels 100 at or relative to the proximal end 44 of the sheath 30 (e.g., when closed, blood or other liquid present in the channels 100 does not flow past the valve assembly 34), and permits selective access into respective ones of the channels 100 by a separate body or device, such as a guidewire, delivery device, etc. The valve assembly 34 can assume various forms appropriate for providing these features. One non-limiting example of the valve assembly 34 is shown in FIGS. 11A and 11B, and includes two or more leaflet-like structures 130. The number of leaflet-like structures 130 corresponds with the number of channels 100, with each leaflet-like structure 130 extending across a corresponding one of the channels 100. With embodiments in which the wire management device 20 includes four of the divider assemblies 32, the four leaflet-like structures 130 are offset 45 degrees from the divider assemblies 32. Regardless, the leaflet-like structures 130 may or may not be inflatable, and can be individually “opened” relative to the corresponding channel 100 (e.g., each of the leaflet-like structures 130 can have a fixed edge affixed to a ring 132 and extend to a free edge; to access the corresponding channel 100, a force is applied to the free edge, causing the leaflet-like structure 130 to deflect, pivoting at the fixed edge). A collapsible sealing body 134 can also be provided that effects a fluid tight seal relative to each of the leaflet-like structures 130. In this way, an individual guidewire can be inserted into a corresponding, individual one of the channels 100 as described above. Further, as generally reflected by FIG. 11B, all of the leaflet-like structures 130 can be simultaneously opened or deflected, as can the optional sealing body 134, for example to accommodate passage of a larger device such as an endograft delivery device (not shown).

With reference to FIGS. 1-11B, the wire management devices of the present disclosure can be useful for a number of different surgical procedures. In some embodiments, for example, the catheter systems of the present disclosure can be used as part of a coronary stent graft or endograft implantation procedure. These and other procedures can be akin to the techniques described, for example, in U.S. Pat. No. 10,398,579, entitled “Catheter System With Guidewire Compartmentalization”, the entire teachings of which are incorporated herein by reference. In general terms, the guidewires 120 are passed separately down each of the channels 100 and then directed by the operator under fluoroscopy into the different visceral branches. The back end of each wire 120 is used to cannulate the corresponding opening in a constrained branched or fenestrated aortic endograft, for example. The constrained endograft 90 is then advanced successfully through the sheath 30, first collapsing the valve assembly 34 forward (into the sheath 30), and then collapsing the vanes 70 of the divider assemblies 32 in the sheath 30 thereby unifying the lumen 40. The constrained endograft 90 can thus be advanced up the sheath 30 collapsing the vanes 70 and uniting the channels 100 as it advances (“unzipping” the partitions) until it reaches the distal end 46 of the sheath 30. Because the vanes 70 stay erect until the endograft collapses them from below, the guidewires 120 stay separated until the point where the endograft reaches them. With the sheath 30 positioned right at the lowest visceral branch, once the device 90 is at the distal end 46 of the sheath 30, the sheath 30 can be pulled back and the device 90 unconstrained partially to allow separation of each of its branches (or exposure of the fenestrations). The device 90 can then be cautiously advanced forward until each of its branches has advanced to the origin of the corresponding visceral branch-its final destination. The endograft can then be completely opened. Bridging stents as necessary can be advanced over each visceral branch wire to make a complete seal. With the endograft deployed, the delivery device 90 is retrieved. After ballooning of the stents to ensure seal, the guidewires 120 can be removed. Lastly the wire management device 20 is removed. As a result, the endograft is completely deployed and wire-wrap is completely averted with the wire management devices of the present disclosure

The deployment procedure described above is but one non-limiting example of a procedure utilizing the wire management devices of the present disclosure. A number of other procedures can be performed that make use of more or less than four guidewires. Multiple other procedures benefiting from subdivision of a tube, such as procedures in a patient's airway or GI tract, are also envisioned by the present disclosure.

Although the present disclosure has been described with reference to preferred embodiments, workers skilled in the art will recognize that changes can be made in form and detail without departing from the spirit and scope of the present disclosure.

What is claimed is:

1. A wire management device comprising:
a sheath defining a lumen and an inner surface;
a first divider assembly projecting from the inner surface;
a second divider assembly projecting from the inner surface;
wherein the first and second divider assemblies provide a normal, extended state in which an edge of the first divider assembly contacts an edge of the second divider assembly to divide the lumen into a plurality of channels;
and further wherein the first and second divider assemblies are collapsible to collapsed state in which the lumen is unified; and
a valve device closing the channels relative to a proximal end of the sheath and configured to permit selective access to individual ones of the channels.

2. The wire management device of claim 1, wherein the first and second divider assemblies each comprises a plurality of vanes.

3. The wire management device of claim 1, wherein the first and second divider assemblies are feather-like.

4. The wire management device of claim 1, wherein the first and second divider assemblies are biased in a direction of a distal end of the sheath.
